(19) United States
(12) Patent Application Publication
(10) Pub. No.: US 2013/0157956 A1
(43) Pub. Date: Jun. 20, 2013

(54) BIOCOMPATIBLE COMPOSITIONS FOR TISSUE AUGMENTATION

(71) Applicants: Sebastianus Gerardus Kluijtmans, Tilburg (NL); Annemarie Tuin, Tilburg (NL); Marja Johanna Adriana Van Luyn, Tilburg (NL); Martin Conrad Harmsen, Tilburg (NL); Jan Bastiaan Bouwstra, Tilburg (NL)

(72) Inventors: Sebastianus Gerardus Kluijtmans, Tilburg (NL); Annemarie Tuin, Tilburg (NL); Marja Johanna Adriana Van Luyn, Tilburg (NL); Martin Conrad Harmsen, Tilburg (NL); Jan Bastiaan Bouwstra, Tilburg (NL)

(73) Assignee: FUJIFILM MANUFACTURING EUROPE B.V., Tilburg (NL)

(21) Appl. No.: 13/818,760

(22) PCT Filed: Aug. 12, 2011
(86) PCT No.: PCT/GB11/51521

§ 371 Date: Feb. 25, 2013

(30) Foreign Application Priority Data
Aug. 31, 2010 (GB) 1014388.1

(51) Int. Cl. A61L 27/26; A61L 27/52; A61L 27/54

(52) U.S. Cl. CPC A61L 27/26; A61L 27/54; A61L 27/52
USPC 514/17.2

(57) ABSTRACT

A crosslinked gelatin hydrogel composition for tissue augmentation.

### FIELD OF INVENTION

The present invention relates to compositions suitable for use in various tissue augmentation procedures.

### BACKGROUND OF THE INVENTION

The present invention relates to compositions for soft tissue augmentation by subcutaneous or intradermal injection. The compositions of the invention are intended to be used in humans in reparative or plastic surgery and in esthetic dermatology, for filling wrinkles, fine lines, skin cracks, acne scars and other scars, as well as in dentistry for filling the gums. Up to now, a number of products have been used for this purpose. These products can be divided in two groups, the so-called permanent tissue augmentation materials and the temporary tissue augmentation materials. Permanent tissue augmentation materials comprise biocompatible polymers that are do not degrade inside the human body. Examples of such products are silicone gel (or silicone oil) and polytetrafluorenthylene particles. However, these permanent materials are associated with chronic inflammation, the formation of granulomas, and even of tardive allergic reactions. Because these permanent tissue augmentation materials are not biodegradable, they are often found in the liver. Temporary tissue augmentation materials, on the contrary, comprise biocompatible polymers that eventually degrade within the human body. Depending on the rate of degradation of the biocompatible polymer these treatments must be repeated within 3 to 12 month to maintain the cosmetic effect. In this group of tissue augmentation materials several different biopolymers are used. Collagen suspensions have been used. The results have however been quite disappointing since collagen is resorbed within 1 to 6 months. Moreover allergic reactions are also noted in about 2% of patients. The collagen used is often of bovine origin, which poses a medical risk in view of a possible allergic response and prion associated diseases. Another biopolymer, which has superseeded the use of collagen is hyaluronic acid, usually crosslinked. Currently 6 hyaluronic acid based tissue augmentation products are FDA approved and on the market in the US, they are: Restylane® (Medicis, Scottsdale, Ariz.), Perlane® (Medicis, Scottsdale, Ariz.), Prevelle Silk® (Mentor Corp., Santa Barbara, Calif.), Hylaform Plus® (Allergan, Irvine, Calif.), Anika® (Anika Therapeutics, Inc., Mass.), and Juvéderm0 (Allergan, Inc., Irvine, Calif.) (Table 1). Juvederm (Allergan, Inc., Irvine, Calif.), which is also known as Hydrafill®, was approved by the FDA in June 2006 for the correction of moderate to severe facial wrinkles and folds. Juvéderm filler agents have been on the market in European countries and Canada since 2003 (marketed as Juvederm by the Corneal Group and by Allergan, formerly Inamed, and in some countries as Hydra Fill® by Allergan, formerly named).

Features that differentiate the various hyaluronic acid based tissue augmentation products are particle size, the type of crosslinking agent used, the degree of crosslinking, the percentage of cross-linked hyaluronic acid, the amount of free (unmodified) hyaluronic acid present, and G′ (elastic modulus). All these physical and chemical attributes influence the clinical characteristics of each filler, such as clinical indication, ease of injection, degree of tissue filling, longevity, clinical appearance, and side effects.

The aim of the invention is to overcome the disadvantages of the current products, by providing an improved longevity of the filling effect without inflammatory and other undesirable side effects.

### SUMMARY OF THE INVENTION

The present invention relates to an extrudable and biocompatible composition for tissue augmentation comprising crosslinked recombinant gelatin-like protein and crosslinked hyaluronic acid wherein the recombinant gelatin-like protein is free from homo- or heterotrimeric structures and wherein the mass ratio between the recombinant gelatin-like protein and the hyaluronic acid is in the range of from 60:40 to 5:95 and wherein the in vivo degradation time of the composition is at least 1 month

### General Definitions

The term “comprising” is to be interpreted as specifying the presence of the stated parts, steps or components, but does not exclude the presence of one or more additional parts, steps or components.

Reference to an element by the indefinite article “a” or “an” does not exclude the possibility that more than one of the element is present, unless the context clearly requires that there be one and only one of the elements. The indefinite article “a” or “an” thus usually means “at least one”.

The term “augmentation” as used herein means the repair, decrease, reduction or alleviation of at least one symptom or defect attributed due to loss or absence of tissue, by providing, supplying, augmenting, or replacing such tissue with the compositions of the present invention.

The term “tissue augmentation” as used herein includes, but is not limited to, the following: dermal tissue augmentation; filling of lines, folds, wrinkles, minor facial depressions, cleft lips and the like, especially in the face and neck; correction of minor deformities due to aging or disease, including in the hands and feet, fingers and toes; augmentation of the vocal cords or glottis to rehabilitate speech; dermal filling of sleep lines and expression lines; replacement of dermal and subcutaneous tissue lost due to aging; lip augmentation; filling of crow's feet and the orbital groove around the eye; breast augmentation; chin augmentation; augmentation of the cheek and/or nose; bulking agent for periurethral support, filling of indentations in the soft tissue, dermal or subcutaneous, due to, e.g., overzealous liposuction or other trauma; filling of acne or traumatic scars and rhytids; filling of nasolabial lines, nasoglabellar lines and infraoral lines. Moreover, the present invention can be directed to hard tissue augmentation. The term ‘hard tissue’ includes but is not limited to bone, cartilage and ligament.

The terms “protein” or “polypeptide” or “peptide” are used interchangeably and refer to molecules consisting of a chain of amino acids, without reference to a specific mode of action, size, three-dimensional structure or origin.

“Gelatin” and “gelatine-like protein” as used herein refers to any gelatin, whether extracted by traditional methods or recombinant or biosynthetic in origin, or to any molecule having at least one structural and/or functional characteristic of gelatin. The term encompasses both the composition of more than one polypeptide included in a gelatin product, as well as an individual polypeptide contributing to the gelatin material. Thus, the term gelatin as used in reference to the present invention encompasses both a gelatin material comprising gelatin polypeptides, as well as an individual gelatin polypeptide. Polypeptides from which gelatin can be derived are polypeptides such as collagens, procollagens, and other polypeptides having at least one structural and/or functional characteristic of collagen. Such a polypeptide could include a single collagen chain, or a collagen homotrimer or heterotrimer, or any fragments, derivatives, oligomers, polymers, or subunits thereof, containing at least one collagenous domain (Gly-X-Y region). The term “recombinant gelatin-like protein” specifically contemplates engineered sequences not found in nature, such as altered collagen sequences, e.g. a sequence that is altered, through deletions, additions, substitutions, or other changes, from a naturally occurring collagen sequence. Such sequences may be obtained from, for example, suitable altered collagen polynucleotide constructs.

A “cross-linking agent” as described herein refers to a composition comprising a cross-linker. “Cross-linker” as used herein refers to a reactive chemical compound that is able to introduce covalent intra- and inter-molecular bridges in organic molecules.

A “hydrogel” refers to a network of polymer chains comprising a substantial amount of water. Depending on the application, i.e. the desired release profile of the active agent and the mechanical stress to which the hydrogel is to be subjected, several types of hydrogels can be used. For example hydrogels that are very stiff and inelastic containing 40-60% of water, hydrogels that are elastic but still rigid containing 60-85% of water and hydrogels that are soft and very elastic containing 85-99% of water.

“Gel strength” is defined as the force required to deform the hydrogel and is expressed in the so-called storage modulus G′ (unit Pascal, Pa). The gel strength of a hydrogel can be measured by a rheometer. The gel strength may depend on the extent of the deformation as the hydrogel may not only elastically deform but also can break up or re-organize as a result of the deformation. Therefore the measurement of the gel strength is usually performed in oscillatory mode with small oscillations in order to keep the hydrogel intact without destroying the microscopic organization of the gel.

“Extrudable” is defined as being able to eject the hydrogel from a device into the application area. Obviously in order to be extrudable the material should be deformable and flowable. A hydrogel is called injectable when it can be applied to the patient in the need for therapy using suitable injection equipment. For example syringes with fine needles or applicator tips may be used. For filling fine facial wrinkles needles between 20 and 30 Gauge are useful.

The in vivo degradation time is the time for at least 90% of the composition to be degraded inside the body of a living being. Thus at least 90% of material is not identifyable anymore as the original composition compared to the situation just after implantation. The in vivo degradation time may refer to either the degradation of the composition as such or the degradation of the individual components.

A hydrogel is called biphasic when it consists of two well-separated gel phases which are distinguishable in terms of their composition.

### DETAILED DESCRIPTION OF THE INVENTION

The inventors of the current invention have surprisingly found that a composition comprising of a mixture of crosslinked recombinant gelatin-like protein and crosslinked hyaluronic acid, wherein the mass ration between these two constituents within in a specific range and an in vivo degradation time, of at least 1 month are effective in tissue augmentation applications such as, for example, use as a dermal filler. The inventors believe, without being bound to theory, that the combined use of recombinant gelatin-like proteins, that are readily absorbed by the body, and hyaluronic acid, which is less susceptible to bioabsorption, leads to the active attraction of fibroblast and fibroblast ingrowth in the injected material. These fibroblasts deposit autologous collagen which aids and prolongs the filling effect of the composition.

Preferably the elastic modulus of the composition of the present invention G′ is at least 50 Pa and more preferably G′ is at least 100 Pa to make it injectable without having adverse effect on the possible firmness of the augmented area and dispersal of material into surrounding tissues.

It is especially preferred that the composition of the present invention has a gel strength, G′, of at least 100 Pa and an in vivo degradation time of at least 3 months.

The composition of the present invention comprises recombinant gelatin-like proteins. Gelatin-like in this context means that the sequence of the protein may contain modifications leaving a sequence consisting of Gly-Xaa-Yaa (Xaa and Yaa may be any amino acid) not completely intact but without affecting the otherwise structural and functional properties of a gelatin, in particular regarding their biocompatibility.

The use of recombinant gelatins is of medical benefit in comparison to the conventionally produced gelatins from animal sources. Safety issues, such as concern over potential immunogenic, e.g., antigenic and allergenic responses, have arisen. Also the inability to completely characterize, purify, or satisfactory reproduce naturally derived gelatin mixtures is of ongoing concern in the pharmaceutical and medical communities. There are also additional safety concerns with respect to bacterial contamination and endotoxin loads resulting from the extraction and purification processes.

Recombinant technology allows the design of gelatin-like proteins with superior characteristics such as, for example, low immunogenicity, improved cell attachment and/or controlled biodegradability. EP 0926543, EP 1014176 and WO 01/34646, and also specifically the examples of EP 0926543 and EP 1014176, describe recombinant gelatins and their production methods, using methylotrophic yeasts, in particular Pichia pastoris.

Another important advantage of recombinant gelatins is that the amino acid sequence can be manipulated to create certain desirable characteristics. Examples of these characteristics are (i) the amount of cross-linkable amino acids (for example the amount of (hydroxy)lysines), (ii) the glycosylation pattern (for example the absence of threonine and/or serine amino acids in certain triplets results in the absence of glycosylation), (iii) the size of the recombinant gelatin, (iv) the charge density of the recombinant gelatin can be amended (for example charged amino acids, such as asparagine (Asn), aspartic acid (Asd), glutamine (Gin), glutamic acid (Glu) or lysine (Lys) can be introduced or left out) or (v) the biodegradability can be amended by the presence or absence of cleavage sites for metalloproteases.

A further embodiment of the present invention provides a composition comprising a recombinant gelatin-like protein wherein the recombinant gelatin-like protein is further enriched in RGD motifs. RGD-enriched gelatins in the context of this invention are described in WO 04/085473 and WO 08/103041 which are incorporated herein by reference.

Preferably in the recombinant gelatin-like prein the percentage of RGD motifs related to the total number of amino acids is at least 0.4 and if the RGD-enriched gelatin comprises 350 amino acids or more, each stretch of 350 amino acids contains at least one RGD motif.

Thus, preferably in the composition of the present invention the recombinant gelatin-like protein comprises at least one RGD motif.

In a further preferred embodiment the recombinant gelatin-like protein is free from hydroxyproline residues. Hydroxylation of prolines is a requirement for the formation of homo- or heterotrimeric triple helices in collagen and plays a role in gelation of gelatin, which is an unfavorable characteristic for the composition of the current invention. In particular less than 10%, more preferably less than 5% of the amino acid residues of the recombinant gelatin-like protein are hydroxyprolines. Preferably the recombinant gelatin-like protein is free from hydroxyprolines. A further benefit described in WO 02/070000 A1 of recombinant gelatins which are free from hydroxyprolines is that they do not show immune reactions involving IgE, in contrast to natural gelatin. It is also preferred that in the compositions of the present invention the recombinant gelatin-like protein is free of hydroxylated amino acid residues.

In a further preferred embodiment the recombinant gelatin-like protein comprises functionalized recombinant gelatin-like proteins for enhanced cell binding and/or with minimal immunogenicity such as, for example, those disclosed in EP 1608681 and EP 1368056 which are incorporated herein by reference. As mentioned above one important characteristic of the recombinant gelatin-like protein is the amount of cross-linkable amino acids, such as the amount of (hydroxy)lysine groups and the amount of carboxylic acid groups derived from aspartic and glutamic acid.

In a preferred embodiment, the invention provides a composition wherein the recombinant gelatin-like protein comprises at least 0.30 mmol/g lysine and/or hydroxylysine (preferably lysine) residues before cross-linking, more preferably at least 0.40 mmol/g, especially at least 0.60 mmol/g and more especially at least 0.80 mmol/g. More preferably the invention provides a composition wherein the recombinant gelatin-like protein comprises at least 0.30 mmol lysine/g and/or hydroxylysine (preferably lysine) residues before cross-linking and at least 0.15 mmol/g free amine groups after cross-linking.

In another further embodiment the recombinant gelatin-like proteins used in the composition of the present invention are recombinant gelatins with an iso-electric point above 5, preferably an iso-electric point above 6 and most preferably an iso-electric point above 7. The objective of this is to provide gelatin-like proteins with a net positive charge gelatin under physiological conditions. Without being bound to theory, this positively charged recombinant gelatin-like protein in the composition of the current invention is thought to aid the attraction, interaction and binding of cells, which have an overall negatively charged membrane. This is a beneficial feature, in tissue augmentation.

A further embodiment provides recombinant gelatin-like proteins used in the compositions of the present invention which have a molecular weight of at least 25 kDa, more preferably of at least 35 kDa and most preferably of at least 50 kDa.

The recombinant gelatin-like protein may be crosslinked using cross-linking agents and techniques such as would be well known to one skilled in the art.

Suitable cross-linking agents include: aldehyde compounds, such as formaldehyde and glutaraldehyde, carbodiimide, di-aldehyde di-isocyanate, ketone compounds such as diacetyl and chloropentanedion, bis (2-chloroethylurea), 2-hydroxy-4,6-dichloro-1,3,5-triazine, reactive halogen-containing compounds disclosed in U.S. Pat. No. 3,288,775, carbamoyl pyridinium compounds in which the pyridine ring carries a sulphate or an alkyl sulphate group disclosed in U.S. Pat. No. 4,063,952 and U.S. Pat. No. 5,529,892, divinylsulfones, and the like and S-triazine derivatives such as 2-hydroxy-4,6-dichloro-s-triazine. In a preferred embodiment the crosslinked recombinant gelatin-like protein of the composition of the current invention is formed by cross-linking with a water soluble carbodimide. In an even more preferred embodiment the crosslinked recombinant gelatin-like protein is cross-linked using 1-ethyl-3-[3-dimethylaminopropyl]carbodiimide hydrochloride (EDC or EDAC). Cross-linking reaction conditions vary depending on which cross-linking agent is used and would be well known to a skilled person.

The crosslinked recombinant gelatin-like protein may be formed by crosslinking more than one form of recombinant gelatin-like protein.

Preferably at least 50 percent (by weight) of the crosslinked recombinant gelatin-like protein comprises at least one RGD motif more preferably at least 70 percent (by weight) of the crosslinked recombinant gelatin-like protein comprises at least one RGD motif and it is especially preferred that at least 90 percent (by weight) of the crosslinked recombinant gelatin-like protein comprises at least one RGD motif

A characteristic of the composition of the present invention is, that the gelatin used should be biodegradable as non-biodegradable compositions for tissue augmentation may lead to the development of granulomas. Moreover biodegradability is another important stimulatory factor in the regeneration of tissue. A priori it is not obvious whether recombinant gelatin-like proteins will be broken down by the same mechanisms causing degradation of natural gelatins. It is known that natural gelatins and collagens are degraded in the human body by proteases and more specifically matrix-metalloproteinases (MMP). Matrix metalloproteinases (MMP's) are zinc-dependent endopeptidases. The MMP's belong to a larger family of proteases known as the metzincin superfamily. Collectively they are capable of degrading all kinds of extracellular matrix proteins, but also can process a number of bioactive molecules. An important group of MMP's are the collagenases. These MMP's are capable of degrading triple-helical fibrillar collagens into distinctive ¾ and ¼ fragments. These collagens are the major components of bone and cartilage, and MMP's are the only known mammalian enzymes capable of degrading them. Traditionally, the collagenases are: MMP-1 (Interstitial collagenase), MMP-8 (Neutrophil collagenase), MMP-13 (Collagenase 3) and MMP-18 (Collagenase 4). Another important group of MMP's is formed by the gelatinases. The main substrates of these MMP's are type IV collagen and gelatin, and these enzymes are distinguished by the presence of an additional domain inserted into the catalytic domain. This gelatin-binding region is positioned immediately before the zinc binding motif, and forms a separate folding unit which does not disrupt the structure of the catalytic domain. The two members of this sub-group are: MMP-2 (72 kDa gelatinase, gelatinase-A) and MMP-9 (92 kDa gelatinase, gelatinase-B). However, International Patent Application WO/2008103045 discloses that a recombinant gelatin that does not comprise a known cleavage site for MMP was enzymatically degradable by human matrix metalloproteinase 1 (MMP1). Apparently many more types of recombinant gelatin than predicted can be degraded. Therefore the composition comprising a crosslinked recombinant gelatin-like protein preferably exhibits the required gradual biodegradation for a composition providing a cellular scaffold function at first instance which is gradually replaced by autologous extracellullar matrix as it degrades.

Hyaluronic acid or hyaluronan is a naturally occurring linear polysaccharide. It can be found in skin, connective, epithelial, and neural tissues. It is ubiquitous across all species and does not require skin allergy testing prior to injection, which makes it very convenient for use in tissue augmentation. This glycosaminoglycan has the ability to bind 1,000 times its volume in water, which makes it the perfect substance for adding volume to the skin. In humans, the amount of naturally occurring hyaluronic acid in the skin decreases with age, which plays an important role in the development of the aging features and wrinkle formation, resulting in decreased tissue elasticity and hydration. Hyaluronic acid can be isolated and prufied from animal derived cartilage tissue such as, for example, rooster combs. However, preferably the hyaluronic acid component is produced by the fermentation of microorganisms that produce hyaluronic such as, for example, bacteria of the genus Streptococcus. More preferably the hyaluronic acid is produced by fermentation of a suitable micro-organism which is genetically engineered to express a recombinant hyaluronic acid synthase.

Unmodified, natural hyaluronic acid has a half-life of approximately 24 hours before it is enzymatically broken down and metabolized in the liver into byproducts, water and carbon dioxide. In the skin, hyaluronic acid is broken down by hyaluronidase and by free radicals.

Hyaluronic acid may be cross linked using cross-linking agents and techniques such as would be well known to one skilled in the art.

Suitable cross-linking agents include: aldehydes, such as formaldehyde and glutaraldehyde, carbodiimide, di-aldehyde di-isocyanate, ketones such as diacetyl and chloropentanedion, bis (2-chloroethylurea), 2-hydroxy-4,6-dichloro-1,3,5-triazine, reactive halogen-containing compounds disclosed in U.S. Pat. No. 3,288,775, carbamoyl pyridinium compounds in which the pyridine ring carries a sulphate or an alkyl sulphate group disclosed in U.S. Pat. No. 4,063,952 and U.S. Pat. No. 5,529,892, divinylsulfones, and the like and S-triazine derivatives such as 2-hydroxy-4,6-dichloro-s-triazine.

In a preferred embodiment the hyaluronic acid is crosslinked by 1,4-butanediol diglycidyl ether (BDDE).

The gelatin and hyaluronic acid components may be mixed prior to crosslinking. However preferably the hyaluronic acid is crosslinked prior to mixing with the gelatin component.

In the composition of the present invention it is preferred that the cross-linked recombinant gelatin-like protein fraction has a higher in vivo degradation time then the crosslinked hyaluronic fraction.

Preferably in the composition of the invention the overall mass ratio of the recombinant gelatin-like protein to hyaluronic acid is preferably in the range of from 30:70 to 5:95, more preferably in the range of from 20:80 to 5:95 and even more preferably in a ratio of at least 10:90.

The composition of the present invention may be homogeneous, i.e. both gelatin and hyaluronic acid are mixed on a molecular level, or inhomogenous. More preferred are Inhomogenous compositions comprising of two or more phases that are characterised by a distinguishable composition. An example of such inhomogenous composition is recombinant gelatin-like protein hydrogel particles in a hyaluronic acid hydrogel continuum. Another example is a bicontinous structure in which a interconnencted hyaluronic acid phase and an interconnected gelatin comprising phase are intermixed. Yet another example is a homogeneous or inhomogeneous gelatin-hyaluronic acid hydrogel phase that is mixed with a hyaluronic hydrogel acid phase. As such the distinguisable phases may consist of pure recombinant gelatin-like protein or hyaluronic acid but can also be homogeneous or inhomogenous mixtures of recombinant gelatin-like protein and hyaluronic acid. The mixing ratio of the various phases can be varied depending on the desired functional characteristics of the overall hydrogel with respect to fibroblast activity and biodegradation.

Preferably the composition of the present invention is biphasic wherein one phase comprises crosslinked recombinant gelatin-like protein and the other phase comprises crosslinked hyaluronic acid.

A second aspect of the invention provides a method for preparing a biocompatible composition according to the first aspect of the invention.

Preferably the composition of the first aspect of the invention is produced using the method comprising:
(a) crosslinking a hyaluronic acid composition to give a hydrogel;
(b) adding a solution comprising gelatin and a gelatin crosslinking agent to the hyaluronic acid hydrogel;
(c) mixing the hydrogel and the gelatin and gelatin crosslinking agent solution;
(d) allowing the mixture to crosslink and form a biphasic hydrogel.

A third aspect of the invention provides the use of a composition as described in the first aspect of the invention for tissue augmentation. Preferably, the composition as described in the first aspect of the invention is used for the manufacture of a medicament for tissue augmentation.

A fourth aspect of the invention provides the use of a composition as described in the first aspect of the invention as a dermal filler. Preferably, the composition as described in the first aspect of the invention is used for the manufacture of a medicament for use as a dermal filler.

A fifth aspect of the invention provides the use of a composition as described in the first aspect of the invention as a localised fibroblast attractant and inducer of deposition of autologous extracellullar matrix in vivo. Preferably, the composition as described in the first aspect of the invention is used for the manufacture of a medicament for use as a localised fibroblast attractant and inducer of deposition of autologous extracellullar matrix in vivo.

A sixth aspect of the invention provides the use of a composition as described in the first aspect of the invention to induce the deposition of autologous collagen. Preferably, the composition as described in the first aspect of the invention is used for the manufacture of a medicament to induce the deposition of autologous collagen.

Compositions prepared in accordance with the present invention may also find use, for example, in other tissue augmentation applications such as for dermal fold augmentation, soft tissue void filling, soft tissue bleb creation, urethral sphincter augmentation for treatment of urinary incontinence, treatment of unilateral vocal fold paralysis, and lower oesophageal sphincter augmentation for treatment of gastroesophageal reflux disease. In some embodiments, the presently described biocompatible compositions may serve as bone void fillers.

The invention will be explained in more detail in the following, non-limiting examples in which all parts and percentages are by weight unless otherwise stated.

### EXAMPLES

### Preparation of Recombinant Gelatin Like Protein/HA Gels

In order to ensure a low bioburden all gels and solutions were prepared in a laminar flow cabinet. Solutions were filtered through a 0.2 μm filter and glassware was autoclaved prior to use.

Gels 1, 2 and 3 are crosslinked pure hyaluronic acid (HA) gels.

Gel 1 was obtained by crosslinking a 10% HA solution (hyaluronic acid salt from Streptococcus equinus sp. Fluka #1351058) in 1% sodium hydroxide using 1,4-butanediol diglycidyl ether (BDDE, Fluka). BDDE was used at 0.5 μmol BDDE/gHA. The gel was allowed to crosslink for 24 hours at room temperature. After neutralizing with 1 M hydrochloric acid the gel was diluted with saline sodium phosphate buffer pH 7.4 to 2.6% and dialysed against saline sodium phosphate buffer for 48 hours. Finally the gel was mashed using a micronizer (IKA, Ultra turrax T25).

Gel 2 and 3 were obtained by starting from a 12% HA solution using 0.42 μmol BDDE/g HA. Gel 2 was not micronized whereas gel 3 was micronized after preparation

As recombinant gelatin like protein recombinant gelatin CBE3 was used which was prepared as described in International Patent Application WO2008103041, incorporated herein by reference. In order to obtain recombinant gelatine like protein/hyaluronic acid (RG/HA) mixed gels (gels 4 and 5), pure CBE3 solutions were crosslinked separately by N-(3-dimethylaminopropyl)-N′-ethylcarbodiimide hydrochloride (EDC) as follows. The recombinant gelatin gel was prepared starting from a 10% CBE3 solution and crosslinked by 2.1 mmol EDC/g RG at pH 4.7. For gel 5 a more firm gel was prepared starting from a 20% CBE3 solution and crosslinked by 5.3 μmol EDC/g RG at pH 4.7. Both gels were allowed to crosslink for 24 hours and subsequently mashed by micronizing until the particles were small enough to pass through a 25 G needle (particle size measured in methanol 200-250 μm, Malvern Mastersizer, type). The mashed RG gels were dialysed against phosphate buffer for 24 hours resulting in RG gels with respectively 2.0 and 6.7% RG. RG/HA mixed gels 4 and 5 were then obtained by homogenously mixing the RG gels with HA gel 1 in a volume ratio of 3:7 using a spatula.

RG-HA mixed gels 6-8 were prepared by first preparing a 10% HA gel with the same amount of BDDE as gel 1. After 24 hours the pH of the HA gel was lowered by addition of 2.25 ml M HCl/10 g HA gel. Then 20% RG solution (pH 7.4) to which 2 ml 50% EDC/10 g gelatin solution had been added, was prepared. Depending of the final HA/gelatine ratio either 6.01 g, 2.56 g, or 1.28 g was and added to 12.25 g HA gel. The mixed gel was homogenized using the ultra turrax. Subsequently the pH was lowered by the addition of 0.2 ml 0.5 M sulfuric acid to initiate EDC crosslinking. Crosslinking proceeded for 30 minutes at 40° C. The gel was diluted with phosphate buffer pH 7.4 to 40 ml and then dialysed for 3 days against phosphate buffer pH 7.4.

RG-HA mixed gels 9-11 were obtained by mixing gels 6-8 with pure HA gel 3 in volume ratio 3:7, leading to overall gelatine and HA concentrations as described in table 1.

Finally all gels were transferred into syringes and frozen samples were sterilized by gamma radiation (7 kG) while kept frozen on dry ice. The gelatin, HA concentrations, and mass ratio of each gel is summarized in Table 1.

TABLE 1
Summary of gel samples, overall RG, HA concentrations, and
RG/HA mass ratio

| Gel | % HA | % RG | RG/HA ratio | RG fraction | HA fraction |
|---|---|---|---|---|---|
| 1 | 2.5 | — | 0 | 0.00 | 1.00 |
| 2 | 2.9 | — | 0 | 0.00 | 1.00 |
| 3 | 2.9 | — | 0 | 0.00 | 1.00 |
| 4 | 1.9 | 0.6 | 0.30 | 0.24 | 0.76 |
| 5 | 1.9 | 2.0 | 0.50 | 0.51 | 0.49 |
| 6 | 2.1 | 2.1 | 1.00 | 0.50 | 0.50 |
| 7 | 2.2 | 1.0 | 0.45 | 0.31 | 0.69 |
| 8 | 2.2 | 0.5 | 0.22 | 0.19 | 0.81 |
| 9 | 2.6 | 0.62 | 0.24 | 0.19 | 0.81 |
| 10 | 2.6 | 0.27 | 0.10 | 0.09 | 0.91 |
| 11 | 2.6 | 0.14 | 0.05 | 0.05 | 0.95 |
| 12 | 0 | 10 | 100 | 1.00 | 0.00 |

### MTS Cytotoxicity Assay

To assess the cytotoxicity of the hydrogels an MTS assay was done. PK-84 cells (5.000 cells per well) were seeded in a 96-well plate. After 24 hours the medium was replaced with extracts of the gels prepared above. Extracts were prepared by shaking the gels in cell culture medium for 24 hours at 37° C. Then 24 hours after addition of the extracts, the mitochondrial substrate MTS [344,5-dimethylthiazol-2-yl)-5-(3-carboxymethoxyphenyl)-2-(4-sulfophenyl)-2H-tetrazolium] was added. Formation of the soluble formazan product was measured at 490 nm using a spectrophotometer.

### Animals

Animal experiments were carried out with male AO rats (Harlan Nederland, Horst, the Netherlands) weighing 250±10 gram. The animals were housed under standard laboratory conditions with a regular light dark cycle and laboratory chow and acidified water ad lib. All animal experiments were approved by the Local Committee on Animal Experimentation.

In vivo Experimental Setup

Animals were put under isoflurane/N₂O/O₂ anaesthesia and their backs were shaved and disinfected. Subsequently 100 μl of each gel was injected subcutaneously at a different location on the back of the rat. The gels and the surrounding tissue were explanted under general anaesthesia after 2 and 4 weeks (n=3 per time point) and thereafter the rats were killed by cervical dislocation.

### Histochemistry

After explanation, the explants were fixed in 2% glutaraldehyde solution prior to embedding in plastic. Sections of 2 μm were stained with toluidine blue and analyzed by light microscopy.

For the measurement of collagen deposition, 2 μm sections were stained with a picrosirius solution (0.1% sirius-red in saturated aqueous picric acid solution for 24 hours at 50° C.). Subsequently, the sections were washed in running tap-water for 5 minutes, counterstained with Mayer's hematoxylin, dehydrated in graded ethanol, cleared in xylene and mounted in resinous medium.

### Immunohistochemistry

Sections of the explants (5 μm) were cut at −25° C. and fixed with acetone. Thereafter, sections were preincubated with 10% rabbit serum and subsequently incubated with antibodies against respectively CD68 (Serotec Ltd, Oxford UK) for the detection of macrophages and giant cells and collagen IV (cat. no. ab6311-100) for the detection of blood vessels. To determine which collagens are deposited in the gels stainings for collagen I (cat. no. ab6308-100) and collagen III (cat. no. ab6310-100, Abcam) were performed. After incubation with the first antibody, endogenous peroxidase activity was blocked by H₂O₂ treatment which was followed by incubation with appropriate secondary antibodies (DAKO, Glostrup Denmark). Slides were then stained with 3-amino-9-ethylcarbazole (AEC), counterstained with hematoxylin and embedded in Kaisers glycerin.

### Statistical Analysis

All data were expressed as the mean ±SD. The data were analyzed using a two-sided Student's t-test assuming similar variances. Differences are considered significant at p<0.05.

### Results

TABLE 2
Summary of histological analysis results at 4 weeks. Gel samples refer to
table 1.

| | Stroma/tissue | Collagen | | |
|---|---|---|---|---|
| Gel | ingrowth | deposition | vascularization | degradation |
| 1 | +−− | +−− | −−− | −−− |
| 2 | −−− | −−− | −−− | −−− |
| 3 | +−− | −−− | −−− | −−− |
| 4 | +−− | −−− | +−− | −−− |
| 5 | +−− | −−− | +−− | −−− |
| 6 | +++ | +++ | +++ | ++− |
| 7 | +++ | +++ | +++ | ++− |
| 8 | ++− | ++− | +++ | +−− |
| 9 | ++− | ++− | ++− | +−− |
| 10 | ++− | ++− | ++− | −−− |
| 11 | ++− | ++− | ++− | −−− |
| 12 | +++ | +++ | +++ | +++ |

−−− = negligible,
+−− = small,
++− = significant,
+++ = strong

Clearly addition of recombinant gelatin like proteins to HA gels has a profound effect on the extent of tissue ingrowth (mainly fibroblasts), vascularization, and collagen deposition inside the gel, and the degradation speed of the gel. In general addition of recombinant gelatin like proteins induces stronger tissue ingrowth, vascularization and autologous collagen deposition, and increases the degradation speed. These effects also increase with increasing gelatin amount. It is noticable that biphasic mixtures such as gels 9 to 11 show beneficial behaviour as the stroma-like tissue ingrowth, vascularization and collagen deposition is strongly increased compared to pure HA gels while only a little faster degradation was observed after 4 weeks. The latter being especially suited for prolonged dermal filling purposes.

1. A biocompatible composition for tissue augmentation comprising crosslinked recombinant gelatin-like protein and crosslinked hyaluronic acid, wherein the recombinant gelatin-like protein is free of homo- or heterotrimeric structures and wherein the mass ratio between the recombinant gelatin-like protein and the hyaluronic acid is in the range of from 60:40 to 5:95 and wherein the in vivo degradation time of the composition is at least 1 month.

2. The composition according to claim 1 wherein the recombinant gelatin-like protein comprises at least 0.3 mmol/g lysine and/or hydroxylysine residues before cross-linking and at least 0.15 mmol/g free amines after cross-linking.

3. The composition according to claim 1 wherein the recombinant gelatin-like protein is free of hydroxylated amino acid residues.

4. The composition according to claim 1 wherein the recombinant gelatin-like protein comprises at least one RGD motif.

5. The composition according to claim 1 wherein the overall mass ratio of the recombinant gelatin-like protein to hyaluronic acid is at least 10:90.

6. The composition according to claim 1 wherein the cross-linked recombinant gelatin-like protein fraction has a higher in vivo degradation time then the crosslinked hyaluronic fraction.

7. The composition according to claim 1 wherein the composition is biphasic wherein one phase comprises crosslinked recombinant gelatin-like protein and the other phase comprises crosslinked hyaluronic acid.

8. A method for preparing a composition as described in claim 1 comprising:
(a) crosslinking a hyaluronic acid composition to give a hydrogel;
(b) adding a solution comprising recombinant gelatin-like protein and a gelatin crosslinking agent to the hyaluronic acid hydrogel;
(c) mixing of the hydrogel and the gelatin and a gelatin crosslinking agent solution; and
(d) allowing the mixture to crosslink and form a biphasic hydrogel.

9. The use of a composition as described in claim 1 as a dermal filler.

10. The use of a composition as described in claim 1 as a localised fibroblast attractant

11. The use of a composition as described in claim 1 to induce the deposition of autologous collagen.
